SEQUENCE LISTING (29 sequences)

SEQ ID NO: 1 - protein, 15 aa, Artificial Sequence
  5D6 CDRL1
RASQSVSTSSYSYMH

SEQ ID NO: 2 - protein, 7 aa, Artificial Sequence
  5D6 CDRL2
YASNLES

SEQ ID NO: 3 - protein, 9 aa, Artificial Sequence
  5D6 CDRL3
QHSWEIPWT

SEQ ID NO: 4 - protein, 5 aa, Artificial Sequence
  5D6 CDRH1
SYWMH

SEQ ID NO: 5 - protein, 17 aa, Artificial Sequence
  5D6 CDRH2
DIYPGTDSTNYNEKFKS

SEQ ID NO: 6 - protein, 7 aa, Artificial Sequence
  5D6 CDRH3
SGDYYDY

SEQ ID NO: 7 - protein, 15 aa, Artificial Sequence
  9E1 CDRL1
RASQSVSTSSYSYMH

SEQ ID NO: 8 - protein, 7 aa, Artificial Sequence
  9E1 CDRL2
YASNLES

SEQ ID NO: 9 - protein, 9 aa, Artificial Sequence
  9E1 CDRL3
QHSWEIPPT

SEQ ID NO: 10 - protein, 5 aa, Artificial Sequence
  9E1 CDRH1
SYWMH

SEQ ID NO: 11 - protein, 17 aa, Artificial Sequence
  9E1 CDRH2
SIYPGNTDTNYNQKFKG

SEQ ID NO: 12 - protein, 9 aa, Artificial Sequence
  9E1 CDRH3
YGTSEGFAY

SEQ ID NO: 13 - protein, 111 aa, Artificial Sequence
  5D6 Light chain variable region
DIVLTQSPASLAVSLGQRATISCRASQSVSTSSYSYMHWYQQKPGQPPKLLIKYASNLESGVPARFSGSGSGTDFTLNIHPVEEEDTATYYCQHSWEIPWTFGGGTKLEIK

SEQ ID NO: 14 - protein, 116 aa, Artificial Sequence
  5D6 Heavy chain variable region
EVQLQQSGAELVKPGTSVKMSCKASGYTFTSYWMHWVRQRPGQGLEWIGDIYPGTDSTNYNEKFKSKATLTVDTSSSTAYMQLSSLTSEDSAVYYCSRSGDYYDYWGQGTTVTVSS

SEQ ID NO: 15 - protein, 111 aa, Artificial Sequence
  9E1 Light chain variable region
DIVMTQSPASLAVSLGQRATISCRASQSVSTSSYSYMHWYQQKPGQPPKLLIKYASNLESGVPARFSGSGSGTDFTLNIHPVEEEDSATYYCQHSWEIPPTFGGGTKLEIK

SEQ ID NO: 16 - protein, 118 aa, Artificial Sequence
  9E1 Heavy chain variable region
EVQLQQSGTVLARPGASVKMSCRASGYSFTSYWMHWVKQRPGQGLEWIGSIYPGNTDTNYNQKFKGKAKLTAVTSASTAYMELSSLTNEDSAVYYCTRYGTSEGFAYWGQGTLVTVSA

SEQ ID NO: 17 - protein, 15 aa, Artificial Sequence
  5D6 epitope
EALLKILGDMQPGDY

SEQ ID NO: 18 - protein, 11 aa, Artificial Sequence
  5D6 epitope
EFSITCLVDEE

SEQ ID NO: 19 - protein, 13 aa, Artificial Sequence
  5D6 epitope
IGFEVSDIHPGSD

SEQ ID NO: 20 - protein, 15 aa, Artificial Sequence
  5D6 epitope
HNNGAGLIDGAYTDY

SEQ ID NO: 21 - protein, 6 aa, Artificial Sequence
  5D6 epitope
IAVEWE

SEQ ID NO: 22 - protein, 4 aa, Artificial Sequence
  9E1 epitope
ISDF

SEQ ID NO: 23 - protein, 12 aa, Artificial Sequence
  9E1 epitope
NTQRLPDRVTGV

SEQ ID NO: 24 - protein, 1143 aa, Homo sapiens
  PEPTIDE (1)..(27): Human ITIH1 fused to Fc_ ITIH1 Signal peptide
  PEPTIDE (28)..(912): Human ITIH1 fused to Fc_ ITIH1 (propeptide- chain-propeptide)
  PEPTIDE (913)..(1143): Human ITIH1 fused to Fc_ Fc(Hinge, CH2, CH3 of Immunoglobulin gamma-1 heavy chain)
MDGAMGPRGLLLCMYLVSLLILQAMPALGSATGRSKSSEKRQAVDTAVDGVFIRSLKVNCKVTSRFAHYVVTSQVVNTANEAREVAFDLEIPKTAFISDFAVTADGNAFIGDIKDKVTAWKQYRKAAISGENAGLVRASGRTMEQFTIHLTVNPQSKVTFQLTYEEVLKRNHMQYEIVIKVKPKQLVHHFEIDVDIFEPQGISKLDAQASFLPKELAAQTIKKSFSGKKGHVLFRPTVSQQQSCPTCSTSLLNGHFKVTYDVSRDKICDLLVANNHFAHFFAPQNLTNMNKNVVFVIDISGSMRGQKVKQTKEALLKILGDMQPGDYFDLVLFGTRVQSWKGSLVQASEANLQAAQDFVRGFSLDEATNLNGGLLRGIEILNQVQESLPELSNHASILIMLTDGDPTEGVTDRSQILKNVRNAIRGRFPLYNLGFGHNVDFNFLEVMSMENNGRAQRIYEDHDATQQLQGFYSQVAKPLLVDVDLQYPQDAVLALTQNHHKQYYEGSEIVVAGRIADNKQSSFKADVQAHGEGQEFSITCLVDEEEMKKLLRERGHMLENHVERLWAYLTIQELLAKRMKVDREERANLSSQALQMSLDYGFVTPLTSMSIRGMADQDGLKPTIDKPSEDSPPLEMLGPRRTFVLSALQPSPTHSSSNTQRLPDRVTGVDTDPHFIIHVPQKEDTLCFNINEEPGVILSLVQDPNTGFSVNGQLIGNKARSPGQHDGTYFGRLGIANPATDFQLEVTPQNITLNPGFGGPVFSWRDQAVLRQDGVVVTINKKRNLVVSVDDGGTFEVVLHRVWKGSSVHQDFLGFYVLDSHRMSARTHGLLGQFFHPIGFEVSDIHPGSDPTKPDATMVVRNRRLTVTRGLQKDYSKDPWHGAEVSCWFIHNNGAGLIDGAYTDYIVPDIFEPKSSDKTHTSPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 333 bp, Artificial Sequence
  Nucleic acid encoding 5D6 LC Variable Region
gatattgtgctgacccaatctcctgcttccttagctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggatactgcaacatattactgtcagcacagttgggagattccgtggacgttcggtggagggaccaaactggaaatcaaa

SEQ ID NO: 26 - DNA, 348 bp, Artificial Sequence
  Nucleic acid encoding 5D6 HC Variable Region
gaggtccagctgcaacagtctggggctgaactggtgaagcctgggacttcagtgaaaatgtcctgcaaggcttctggctacaccttcaccagctactggatgcactgggtgaggcagaggccgggacaaggccttgagtggattggagatatttatcctggtactgatagtactaactacaatgagaagttcaagagcaaggccacactgactgtagacacatcctccagcacagcctacatgcaactcagcagcctgacatctgaggactctgcggtctattactgttcaagatcgggagattactacgactactggggccaaggcaccacggtcaccgtctcctca

SEQ ID NO: 27 - DNA, 333 bp, Artificial Sequence
  Nucleic acid encoding 9E1 LC Variable Region
gatattgtgatgacccagtctcctgcttccttagctgtatctctggggcagagggccaccatctcatgcagggccagccaaagtgtcagtacatctagctatagttatatgcactggtaccaacagaaaccaggacagccacccaaactcctcatcaagtatgcatccaacctagaatctggggtccctgccaggttcagtggcagtgggtctgggacagacttcaccctcaacatccatcctgtggaggaggaggattctgcaacatattactgtcagcacagttgggagattcctccgacgttcggtggagggaccaaactggaaatcaaa

SEQ ID NO: 28 - DNA, 354 bp, Artificial Sequence
  Nucleic acid encoding 9E1 HC Variable Region
gaggttcagctgcagcagtctgggactgtgctggcaaggcctggggcttccgtgaagatgtcctgcagggcttctggctacagctttaccagctactggatgcactgggtaaaacagaggcctggacagggtctagaatggattggttctatttatcctggaaatactgatactaactacaaccagaagttcaagggcaaggccaaactgactgcagtcacatccgccagcactgcctacatggagctcagcagcctgacaaatgaggactctgcggtctattactgtacaagatatggtacttccgaggggtttgcttactggggccaagggactctggtcactgtctctgca

SEQ ID NO: 29 - protein, 11 aa, Homo sapiens
  PEPTIDE (1)..(11): Human ITIH1 peptide
DKAREVAFDVE

(19) United States
(12) Patent Application Publication
(10) Pub. No.: US 2022/0403047 A1
(43) Pub. Date: Dec. 22, 2022

(54) ANTIBODY SPECIFICALLY RECOGNIZING ITIH1, AND PHARMACEUTICAL COMPOSITION CONTAINING SAME FOR IMPROVING INSULIN RESISTANCE IN DISEASES ACCOMPANIED BY IMPAIRED GLUCOSE TOLERANCE

(71) Applicant: APHARMA, Goyang-si, Gyeonggi-do (KR)

(72) Inventors: Sang Geon KIM, Seoul (KR); Yeonseok CHUNG, Seoul (KR); Tae Hyun KIM, Seoul (KR); Won KIM, Seoul (KR)

(73) Assignee: APHARMA, Goyang-si, Gyeonggi-do (KR)

(21) Appl. No.: 17/774,250

(22) PCT Filed: Sep. 3, 2020
(86) PCT No.: PCT/KR2020/011805

§ 371 Date: May 4, 2022

(30) Foreign Application Priority Data
Sep. 5, 2019 (KR) 1020190109892

(51) Int. Cl. C07K 16/38; A61P 3/10

(52) U.S. Cl. CPC C07K 16/38; A61P 3/10; A61K 2039/505

(57) ABSTRACT

A pharmaceutical composition for improving insulin sensitivity in diseases accompanied by impaired glucose tolerance is disclosed. The pharmaceutical composition contains an antibody or an antigen-binding fragment thereof that specifically recognizes ITIH1 exhibiting increased expression in diseases accompanying high blood sugar. The pharmaceutical composition can be effectively used for improving insulin sensitivity in various diseases accompanying high blood sugar.

### TECHNICAL FIELD

The present invention relates to an antibody that specifically recognizes ITIH1 and technology for treating diseases accompanied by impaired glucose tolerance using the same.

### BACKGROUND ART

Hyperglycemia accompanied by impaired glucose tolerance is often caused by various types of stressors, toxic stimuli and inflammation that affect cells or tissues, or accompanies the progression of systemic metabolic syndromes including obesity or diabetes. Hyperglycemia is induced by pathological conditions such as excessive glucose production in the liver and decreased glucose utilization in peripheral tissues.

The diseases accompanied by impaired glucose tolerance include metabolic syndromes and diabetes, and more specifically, include a variety of diseases such as metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney disease, muscle disease, and dementia.

Currently, therapeutic agents for diabetes, which is a representative disease accompanied by high blood sugar, and conventional insulin resistance suppressors may be classified into insulin preparations and oral drugs. The oral drugs may be classified into sulfonylureas, which function to promote insulin secretion, and non-sulfonylureas including meglitinides, metformin, which inhibits glucose production in the liver and improves peripheral insulin sensitivity, alpha-glucosidase inhibitors, which inhibit carbohydrate absorption in the intestine, thiazolidinediones, which mainly improve insulin sensitivity, dipeptidyl peptidase (DPP)-4 inhibitors, and the like.

Korean Patent Laid-Open Publication No. 10-2019-0040765 relates to a pharmaceutical composition for preventing or treating diabetes containing a DPP-4 inhibitor and a method for preparing the same.

Korean Patent Laid-open Publication No. 10-2019-0044079 relates to a method of treating severe insulin resistance by interfering with glucagon receptor signaling.

There is a need for development of a novel substance for improving hyperglycemia that targets a novel molecule.

### DISCLOSURE

### Technical Problem

It is an object of the present invention to provide a therapeutic agent targeting ITIH1 for treatment of diseases accompanied by hyperglycemia.

### Technical Solution

In accordance with one aspect of the present invention, the above and other objects can be accomplished by the provision of an antibody or an antigen-binding fragment thereof specifically recognizing an inter-alpha trypsin inhibitor heavy chain 1 (ITIH1).

In one embodiment, the antibody includes a light-chain variable region including the complementarity-determining regions CDRL1, CDRL2, and CDRL3 set forth in SEQ ID NOS: 1, 2, and 3, respectively, and a heavy-chain variable region including the complementarity-determining regions CDRH1, CDRH2, and CDRH3 set forth in SEQ ID NOS: 4, 5, and 6, respectively, or

a light-chain variable region including the complementarity-determining regions CDRL1, CDRL2, and CDRL3 set forth in SEQ ID NOS: 7, 8, and 9, respectively, and a heavy-chain variable region including the complementarity-determining regions CDRH1, CDRH2, and CDRH3 set forth in SEQ ID NOS: 10, 11, and 12, respectively.

In one embodiment, the antibody includes a light-chain variable region set forth in SEQ ID NO: 13 and a heavy-chain variable region set forth in SEQ ID NO: 14; or a light-chain variable region set forth in SEQ ID NO: 15 and a heavy-chain variable region set forth in SEQ ID NO: 16.

In one embodiment, the epitope of ITIH1 recognized by the antibody according to the present invention includes at least one of polypeptides set forth in SEQ ID NO: 17, SEQ ID NO: 18, SEQ ID NO: 19, SEQ ID NO: 20, or SEQ ID NO: 21, for the antibody including the light-chain variable region including the complementarity-determining regions CDRL1, CDRL2, and CDRL3 set forth in SEQ ID NOS: 1, 2, and 3, respectively, and the heavy-chain variable region including the complementarity-determining regions CDRH1, CDRH2, and CDRH3 set forth in SEQ ID NOS: 4, 5, and 6, respectively, or the antibody including the light-chain variable region set forth in SEQ ID NO: 13 and the heavy-chain variable region set forth in SEQ ID NO: 14, and the epitope of ITIH1 recognized by the antibody includes at least one of polypeptides set forth in SEQ ID NO: 22 or SEQ ID NO: 23 for the antibody including the light-chain variable region including the complementarity-determining regions CDRL1, CDRL2, and CDRL3 set forth in SEQ ID NOS: 7, 8, and 9, respectively, and the heavy-chain variable region including the complementarity-determining regions CDRH1, CDRH2 and CDRH3 set forth in SEQ ID NOS: 10, 11, and 12, respectively, or the antibody including the light-chain variable region set forth in SEQ ID NO: 15 and the heavy-chain variable region set forth in SEQ ID NO: 16.

In accordance with another aspect of the present invention, provided is a nucleic acid encoding the antibody according to the present invention, a vector containing the nucleic acid, or a cell including the vector.

In one embodiment, the nucleic acid encoding the light-chain variable region of the antibody has a sequence set forth in SEQ ID NO: 25 or 27, and the nucleic acid encoding the heavy-chain variable region has a sequence set forth in SEQ ID NO: 26 or 28.

In accordance with another aspect of the present invention, provided is a pharmaceutical composition for ameliorating insulin resistance of a disease accompanied by impaired glucose tolerance containing an antibody or antigen-binding fragment that specifically recognizes inter-alpha trypsin inhibitor heavy chain 1 (ITIH1).

Insulin resistance is a symptom of various diseases and diseases accompanied by impaired glucose tolerance exhibiting the symptom include, but are not limited to, metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney diseases, muscle diseases, and dementia. Insulin resistance promotes cell death in the progression of a variety of diseases that may cause inflammation. Therefore, all of the diseases described above are related to insulin resistance.

In addition, amelioration in insulin resistance may provide effects of increasing cell viability and improving cell regeneration based on increased sugar utilization, thus providing anti-inflammatory activity.

In addition, the amelioration in insulin resistance enables effective use as a therapeutic agent for diabetes.

The antibody according to the present invention may be a monoclonal antibody, a chimeric antibody, a humanized antibody, or a human antibody.

In another embodiment, the antibody according to the present invention may be a multimeric antibody, a heterodimeric antibody, a homodimeric antibody, a multivalent antibody, or a single-chain antibody.

In accordance with another aspect of the present invention, provided is a method of ameliorating insulin resistance in a disease accompanied by impaired glucose tolerance, the method including administering the antibody or antigen-binding fragment thereof according to the present invention to a subject in need of amelioration of insulin resistance in a disease accompanied by impaired glucose tolerance.

In one embodiment, the disease accompanied by impaired glucose tolerance in the method includes metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney disease, muscle disease, and dementia.

In another embodiment, the method is used to treat diabetes.

### Advantageous Effects

The pharmaceutical composition according to the present invention contains an antibody specifically recognizing ITIH1, expression of which is increased in diseases accompanied by hyperglycemia, and is capable of ameliorating insulin resistance, that is, remarkably increasing sensitivity when used to neutralize ITIH1, and thus can be effectively used in the treatment of impaired glucose tolerance.

### DESCRIPTION OF DRAWINGS

FIG. 1 shows the result of analysis of variation in the level of O-GlcNAc of ITIH1 in the collected hepatocytes using antibodies of CTD110.6 clones, after primary hepatocytes isolated from the liver of liver-selective Ga13-knockout mice fed a high-lipid diet for 5 weeks were cultured, while 25 mM glucose stimulation was applied to the culture medium for 24 hours, wherein, compared with the low-concentration condition, O-GlcNAc of ITIH1 was increased by stimulation with a high concentration of glucose and the expression of ITIH1 was remarkably increased, and these results indicate that a cell-based assay that mimics the hyperglycemic situation in vivo was successfully established.

FIG. 2 shows the result of western blot analysis of serum ITIH1 expression levels at predetermined times in each of mice (two per group) to which a high concentration of glucose (2 g/kg body weight) was administered. Albumin was used in the same amount as the serum sample and was thus used for comparative evaluation. ITIH1 antibody (Biorbyt, Ltd., UK) and albumin antibody (Cusabio Technology LLC., USA) used as primary antibodies in the western blot experiment were analyzed using commercially available antibodies according to the manufacturer's experimental method. When glucose stimulation was applied, it was found that the content of ITIH1 was increased in the liver tissue and blood of mice, and ITIH1 expression was high in Ga13 gene-deficient mice even in the absence of glucose stimulation compared to normal mice. When glucose stimulation was applied to such mice, the ITIH1 content was very high. This is an effective method that is greatly improved compared to a conventional administration model fed a high-lipid diet for 12 to 16 weeks. The single glucose administration method proposed in the present invention is an experimental method capable of performing target analysis in the corresponding disease situation by easily establishing a hyperglycemia model even through a short-period simple experimental method and thus has advantages of overcoming consumption of a lot of time and money to actually induce hyperglycemia, while consuming a high-fat diet for about 12 to 16 weeks.

FIGS. 3A to 3C show the correlation between the change in the expression of liver Ga13 and blood sugar in various hyperglycemic animal models induced using high-lipid diet, a genetic modification that eliminates an appetite suppression center, and administration of streptozotocin (STZ). As a result, the expression of Ga13 in the liver was decreased in all tested hyperglycemia conditions, indicating that the change in Ga13 expression was directly correlated with hyperglycemia. In addition, the results indicate that the decrease in Ga13 expression and the increase in ITIH1 occur together.

FIG. 4 shows the results of glucose tolerance and insulin tolerance tests performed on hepatocyte-selective Ga13-deficient mice fed a high-lipid diet for 9 to 13 weeks, and shows the state in which fasting blood glucose is significantly increased in the selective Ga13-deficient mice (left), the result of the glucose tolerance test (middle), and the result of the insulin tolerance test (right).

FIG. 5 shows the result of western blot analysis identifying the expression of ITIH1 in liver tissue and sera derived from normal mice or hepatocyte-selective Ga13-deficient mice fed a high-lipid diet, wherein an ITIH1 antibody (Biorbyt, UK), a beta-actin antibody (Sigma, USA) and an albumin antibody (Cusabio, USA) used as primary antibodies in the Western blot experiment were analyzed as commercially available antibodies in accordance with the experimental method suggested by the manufacturer, and FIG. 6 indicates that the expression of ITIH1 was remarkably increased in the livers and sera of Ga13-deficient mice compared to normal mice.

FIG. 6 shows the result of Western blot to determine the amount of ITIH1 in a sample obtained 6 hours after one-time oral administration of a high concentration of glucose (2 g/kg body weight) to normal mice or hepatocyte-selective Ga13-deficient mice, wherein an ITIH1 antibody (Biorbyt, UK), an OGT antibody (Sigma, USA), a Ga13 antibody (Santa Cruz, USA), and a beta-actin antibody (Sigma, USA), used as primary antibodies in the Western blot experiment, were analyzed as commercially available antibodies according to the manufacturer's experimental method, and FIG. 6 shows that the expression of ITIH1 and OGT increased along with the decrease of Ga13 when a high concentration of glucose was administered to normal mice, and the expression was further significantly increased in the livers of Ga13-deficient mice.

FIG. 7 shows the results of glucose and insulin tolerance tests after intraperitoneally administering a polyclonal antibody recognizing ITIH1 at a concentration of 250 μg/kg body weight every day for the last 2 weeks (the 2 weeks prior to the experiment), while providing a high-lipid diet for 11 to 13 weeks to normal or hepatocyte-selective G13-deficient mice. It was found that the glucose and insulin tolerance of the G13-deficient mice was decreased compared to normal mice and the result of administration of the polyclonal antibody neutralizing ITIH1 showed that the glucose and insulin tolerance was significantly ameliorated. In addition, it was found that even when the glucose absorption capability was analyzed in the white fat and skeletal muscle tissues of normal mice fed a high-lipid diet, the glucose absorption capability of the peripheral tissues of the mice administered with the ITIH1 polyclonal antibody was significantly improved. These results indicate that the ITIH1 antibody is effective in ameliorating insulin resistance in hyperglycemia-inducing diseases, metabolic diseases, and diseases characterized by reduced insulin sensitivity.

### MODE FOR INVENTION

The present invention is based on the finding of increased concentration of ITIH1 (inter-alpha trypsin inhibitor heavy chain 1) in liver tissue and blood upon glucose stimulation and the mechanism thereof and on the development of an antibody that specifically recognizes ITIH1. Specifically, the present invention is based on the finding that an increase in OGT (O-GlcNAc transferase) attributable to a decrease in Ga13 (G protein alpha-13) in hyperglycemia increases the stability of ITIH1, which causes an increase in intracellular concentration and secretion thereof, and the finding that antibodies targeting ITIH1 could be effectively used to ameliorate insulin sensitivity in diseases accompanied by impaired glucose tolerance.

In one aspect, the present invention is directed to an antibody or an antigen-binding fragment thereof specifically recognizing an inter-alpha trypsin inhibitor heavy chain 1 (ITIH1), wherein the antibody includes a light-chain variable region including the complementarity-determining regions CDRL1, CDRL2, and CDRL3 set forth in SEQ ID NOS: 1, 2, and 3, respectively, and a heavy-chain variable region including the complementarity-determining regions CDRH1, CDRH2, and CDRH3 set forth in SEQ ID NOS: 4, 5, and 6, respectively, or a light-chain variable region including the complementarity-determining regions CDRL1, CDRL2, and CDRL3 set forth in SEQ ID NOS: 7, 8, and 9, respectively, and a heavy-chain variable region including the complementarity-determining regions CDRH1, CDRH2, and CDRH3 set forth in SEQ ID NOS: 10, 11, and 12, respectively.

In another embodiment, the antibody according to the present invention includes a light-chain variable region set forth in SEQ ID NO: 13 and a heavy-chain variable region set forth in SEQ ID NO: 14, or a light-chain variable region set forth in SEQ ID NO: 15 and a heavy-chain variable region set forth in SEQ ID NO: 16.

The ITIH1 recognized by the antibody according to the present invention may be derived from a variety of sources, and may be capable of recognizing ITIH1 derived from, for example, mammals, especially humans or mice. In addition, even when derived from the same type of host, for example, a human, there may be sequence variations depending on the specific individual, region, environment, etc. All variations, including sequences that have been modified (deleted, substituted, or added) but are functionally equivalent, may be recognized.

In one embodiment, the protein and gene sequences recognized by the antibody according to the present invention are known. For example, it is known that the NCBI (National Center for Biotechnology Information) gene and protein accession numbers for humans are NM_002215.4 and NP_002206.2, and the NCBI gene and protein accession numbers for mice are NM_008406.3 and NP_032432.2. However, the protein and gene sequences of ITIH1 are not limited to the above sequences, and include functional equivalents thereto.

The antibody or antigen-binding fragment thereof according to the present invention may be provided in various forms, as long as it has the characteristics described herein.

As used herein, the term “antibody” refers to a protein that binds to another molecule (antigen) through the variable regions of light and heavy chains, and includes IgG, IgD, IgA, and IgE types. Antibodies include polyclonal antibodies, monoclonal antibodies, and multispecific antibodies. In addition, the antibody of the present invention includes monoclonal antibodies having various types of structures, for example, an intact antibody (intact Ab) including two full-length heavy chains and two full-length light chains, and a fragment thereof, a chimeric antibody, a human antibody, a humanized antibody, or another genetically engineered antibody having characteristics according to the present invention, which includes or does not include a constant region.

As used herein, the term “antigen-binding fragment” refers to a part of the intact antibody described above, and is a sequence having one or more sequences shorter than the amino acid sequence of the intact antibody in length. In terms of functionality, the antigen-binding fragment includes at least a part of the activity or function of the intact antibody or parent antibody, and examples thereof include, but are not limited to, Fab (fragment for antigen binding), Fab′, F(ab′)2, Fv or single-chain antibody (SCA, e.g., scFv or dsFv), bispecific scFv, and diabodies.

The antibodies according to the present invention include antigen-binding fragments, variants, and derivatives thereof, and include, but are not limited to, polyclonal, monoclonal, multispecific, human, humanized, primatized, or chimeric antibodies, single-chain antibodies, and epitope-binding fragments, for example, Fab, Fab′, F(ab′)₂, F(ab)₂, Fd, Fvs, single-chain Fvs (scFV), disulfide-linked Fvs (sdFv), and fragments including a VL or VH region. The antibody according to the preset invention may be of any type, for example IgG, IgE, IgM, IgD, IgA or IgY, and may also be of any class, for example IgG1, IgG2, IgG3, IgG4, IgA1, or IgA2, or a subclass thereof.

The antibody or antibody fragment of the present invention may be a chimeric antibody. As used herein, the term “chimeric antibody” refers to an antibody that includes at least a part of a variable region, that is, an antigen-binding site and a constant region (including CL1 for the light chain, and CH1, CH2, and CH3 for the heavy chain) of the antibody derived from different species. For example, the variable region may be derived from a mouse, and the constant region may be derived from a human. Alternately, the antibody means a class-switched antibody, for example an antibody switched from an IgG type to an IgE type. Chimeric antibodies are typically produced through recombinant DNA techniques, and reference may be made to, for example, Morrison et al. PNAS USA 81 (1984) 6851-6885; and U.S. Pat. No. 5,202,238.

The antibody or antibody fragment of the present invention may be a humanized antibody. As used herein, the term “humanized antibody” means an antibody that has a human antibody as a framework and a portion of the CDR region which has been modified to include only a portion essential for specifically binding to an antigen, among the CDRs of the species from which the antibody molecule is originally derived. For example, among the CDRs of the antibody derived from a monkey or mouse, the remaining CDR regions and light- and heavy-chain frameworks excluding regions essential for specific binding to an antigen are replaced with human antibodies. Production methods are described, for example, in Riechmann et al. (1988) Nature 332:323-327.

The antibody or antibody fragment of the present invention may be a polyclonal or monoclonal antibody. The monoclonal antibody is basically prepared through fusion of myeloma cells with splenocytes derived from immunized mammals, and may be prepared by various methods known in the art.

Furthermore, the antibody, antigen-binding fragment, variant, or derivative thereof according to the present invention may be conjugated to a functional substance such as a therapeutic agent, prodrug, peptide, protein, enzyme, virus, lipid, biological response modifier, or PEG (polyethylene glycol) for various purposes. Depending on the type of the material to be conjugated therewith, it may be prepared using various methods. Reference may be made, for example, to the following literature: Amon et al. “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al. “Antibodies For Drug Delivery”, in Controlled Drug Delivery (2nd Ed.), Robinson et al. (eds.), Marcel Dekker, Inc., pp. 623-53 (1987).

Fragments of antibodies can be obtained by treatment with pepsin or papain. The F(ab′)2 fragment can be obtained by treating an intact antibody with pepsin, which is subsequently treated with a thiol reducing agent to obtain a Fab fragment including part of a light chain and a heavy chain. The Fab fragment may also be obtained by treating an intact antibody with papain. For example, by treating the antibody produced from the hybridoma of the present invention with pepsin or papain, an antibody fragment that specifically recognizes ITIH1 such as F(ab′)₂ or Fab can be prepared.

The Fv fragment is an antibody fragment composed only of the variable regions of the heavy and light chains, and the two variable regions can be linked by non-covalent or covalent bonds such as chemical crosslinking agents or intermolecular disulfide bonds (Inbar et al. (1972) PNAS 69:2659-2662). For example, the Fv fragment may be prepared by treating the antibody produced from the hybridoma of the present invention with an enzyme to isolate only the variable regions of the heavy and light chains, or using recombinant DNA technology.

The SCA fragment may be produced by enzyme treatment or genetic engineering, and is an antibody fragment in which the variable region of the light chain is linked to the variable region of the heavy chain by a linker such as a polypeptide. For the method of producing ScFv, reference may be made, for example, to those described in U.S. Pat. Nos. 4,936,778 or 5,892,019, and the antibody may be produced by treating the antibody produced in the hybridoma according to the present invention with an enzyme or using recombinant DNA technology, for example, producing a vector including a nucleic acid sequence encoding the heavy-chain and/or light-chain variable region of the antibody, and expressing the same in appropriate cells.

As used herein, the term “binding” or “specific binding” refers to the affinity of an antibody or antibody composition of the present invention for an antigen. The term “specific binding” in the antigen-antibody binding may be distinguished from non-specific background binding, typically when the dissociation constant (Kd) is less than 1×10⁻⁵ M, less than 1×10⁻⁶ M, or less than 1×10⁻⁷ M. The specific binding can be detected using a method known in the art, for example, ELISA, surface plasmon resonance (SPR), immunoprecipitation, or coprecipitation, and an appropriate control group that can differentiate the specific binding from the non-specific binding may be present.

The antibody clone 5D6 prepared in one embodiment according to the present invention has a dissociation constant of 2.43×10⁻¹⁰ M, and the other antibody clone 9E1 has a dissociation constant of 1.33×10⁻¹⁰ M, indicating high affinity for ITIH1.

The antibody of the present invention including the intact antibody or fragment thereof as described above may be present as a multimer such as a dimer, trimer, tetramer, or pentamer, which includes at least some of the antigen-binding ability of the monomer. Such multimers are also intended to include homomultimers or heteromultimers. The antibody multimer includes a plurality of antigen-binding sites and thus has superior antigen-binding ability compared to monomers. The multimer of the antibody also enables easy production of a multifunctional (bifunctional, trifunctional, or tetrafunctional) antibody.

As used herein, the term “multifunctional” refers to an antibody or antibody composition having two or more activities or functions (e.g., antigen-binding ability, enzymatic activity, or ligand- or receptor-binding ability). For example, the antibody of the present invention can be bound to a polypeptide having enzymatic activity, for example, luciferase, acetyltransferase, galactosidase, or the like.

The multifunctional antibody also includes a multivalent or multispecific (bispecific, trispecific, etc.) antibody. The term “multispecific” is intended to include, for example, “bispecific”, meaning variable regions capable of binding to two or more different epitopes. The two or more epitopes may be present on one antigen, or may be present on a different antigen.

The antibody of the present invention may be present in intact cells including hybridomas, or a cell lysate or medium thereof, and may be purified and isolated therefrom in a partially or substantially pure form. Purification is performed in order to remove byproducts of cells other than antibodies, such as cell components, nucleic acids, and proteins, and is performed using known methods such as alkaline/SDS treatment, CsCl separation, column chromatography, and agarose electrophoresis. For example, reference may be made to Ausubel et al. (eds), Current Protocols in Molecular Biology, Greene Publishing and Wiley Interscience, New York, latest edition.

In one embodiment, the antibody according to the present invention is prepared using the human ITIH1 sequence (SEQ ID NO: 24) fused to Fc as an antigen.

In one embodiment, the epitope of the antibody according to the present invention includes at least one of polypeptides set forth in SEQ ID NO: 17, SEQ ID NO: 18, SEQ ID NO: 19, SEQ ID NO: 20, or SEQ ID NO: 21 for the antibody including the light-chain variable region including the complementarity-determining regions CDRL1, CDRL2, and CDRL3 set forth in SEQ ID NOS: 1, 2, and 3, respectively, and the heavy-chain variable region including the complementarity-determining regions CDRH1, CDRH2, and CDRH3 set forth in SEQ ID NOS: 4, 5, and 6, respectively, or the antibody including the light-chain variable region set forth in SEQ ID NO: 13 and the heavy-chain variable region set forth in SEQ ID NO: 14, and the epitope of the antibody includes at least one of polypeptides set forth in SEQ ID NO: 22 or SEQ ID NO: 23 for the antibody including the light-chain variable region including the complementarity-determining regions CDRL1, CDRL2, and CDRL3 set forth in SEQ ID NOS: 7, 8, and 9, respectively, and the heavy-chain variable region including the complementarity-determining regions CDRH1, CDRH2, and CDRH3, set forth in SEQ ID NOS: 10, 11, and 12, respectively, or the antibody including the light-chain variable region set forth in SEQ ID NO: 15 and the heavy-chain variable region set forth in SEQ ID NO: 16.

In another aspect, the present invention is directed to a polynucleotide or nucleic acid molecule encoding all or part of the antibody or antigen-binding fragment according to the present invention, a vector including the polynucleotide, a cell including the vector, or a transformant.

The nucleic acid includes, for example, DNA, cDNA, RNA, or recombinant or synthesized DNA or RNA. In one embodiment, the nucleic acid molecule is cDNA. The nucleic acid may also be corresponding genomic DNA or a fragment thereof. A nucleic acid sequence encoding the antibody according to the present invention or a part or fragment thereof may be different due to redundancies in the nucleic acid sequence encoding the amino acid, and such sequences also fall within the scope of the present invention. In one embodiment according to the present invention, the polynucleotide encoding the light-chain variable region of the antibody according to the present invention has a sequence set forth in SEQ ID NO: 25 or 27, and the nucleic acid encoding the heavy-chain variable region has a sequence set forth in SEQ ID NO: 26 or 28.

In another aspect, the present invention is directed to a vector containing and being capable of expressing a nucleic acid molecule. Vectors that may be used in the present invention include, for example, phages, plasmids, and replicable or non-replicable viral or retroviral vectors. Nucleic acid molecules according to the present invention may be introduced into various known vectors. Examples of vectors include vectors for prokaryotic cells such as pUC-based vectors, pBluescript (Stratagene), pET-based vectors (Novagen) or pCRTOPO (Invitrogen) vectors, and vectors for eukaryotic cells, such as pREP (Invitrogen), pcDNA3 (Invitrogen), pCEP4 (Invitrogen), pMCI neo (Stratagene), pXT1 (Stratagene), pSG5 (Stratagene), EBO-pSV2neo, pBPV-1, pdBPVMMTneo, pRSVgpt, pRSVneo, pSV2-dhfr, plZD35, pLXIN, pSIR (Clontech), pIRES-EGFP (Clontech), pEAK-10 (Edge Biosystems), pTriEx-Hygro (Novagen), and pCINeo (Promega) vectors, and the like, but are not limited thereto.

The vector according to the present invention may be introduced into various known prokaryotic or eukaryotic cells by known transformation or transfection methods. When the vector is introduced into cells, it may be inserted into the genome of host cells, or may be present in the form of an extra chromosome.

The prokaryotic cells that can be used include cells of the genera Escherichia, Bacillus, Streptomyces and Salmonella, and the eukaryotic cells include mammalian cells such as Hela, HEK293, H9, Jurkat, mouse NIH3T3, C127, Cos1, Cos7 and CV1, and mouse C2C12, BHK and CHO cells; fungal cells such as Saccharomyces cerevisiae or Pichia pastoris, and insect cells such as Drosophila S2 and Spodoptera Sf9, but the present invention is not limited thereto.

The antibodies of the present invention may be produced by known methods using recombination. In accordance with the recombinant method, the nucleic acid sequence encoding the heavy chain of the antibody according to the present invention and the antibody encoding the light chain of the antibody are cloned into one or two expression vectors, then transfected into eukaryotic host cells to express the antibody, after which the antibodies can be obtained from the host cells or medium. Recombinant methods including the production of the vectors, expression of proteins from the produced vectors in cells, isolation of proteins from the produced vectors are known in the art, and reference may be made, for example, to Kaufman, R. J., Mol. (2000) Biotechnol. 16:151-160, and the like. The vector encoding the antibody of the present invention may be expressed in appropriate host cells, for example, CHO cells, NS0 cells, SP2/0 cells, HEK293 cells, COS cells, yeast, or E. coli, and the antibody may be obtained from a cell lysate or medium.

The expression of antibodies in NS0 cells may be performed with reference to, for example, Barnes et al. (2000) Cytotechnology 32:109-123 and Norderhaug et al. (1997) J. Immunol. Methods 204:77-87, et al. Expression in HEK cells may be performed with reference to Schlaeger, E.-J. (1996) J. Immunol. Methods 194:191-199, et al.

The antibody of the present invention may be present in intact cells including hybridomas, or a cell lysate or medium thereof, and may be purified and isolated therefrom in a partially or substantially pure form. Purification is performed in order to remove byproducts of cells other than antibodies, such as cell components, nucleic acids, and proteins, and is performed using known methods such as alkaline/SDS treatment, CsCl separation, column chromatography, and agarose electrophoresis. For example, reference may be made to Ausubel et al. (eds), Current Protocols in Molecular Biology, Greene Publishing and Wiley Interscience, New York, latest edition.

Monoclonal antibodies may, for example, be obtained by culturing the hybridoma cells disclosed herein and then isolating the same using a conventional method, such as protein A-sepharose, hydroxyapatite chromatography, dialysis, or affinity chromatography.

In another aspect, the present invention is directed to a pharmaceutical composition for ameliorating insulin resistance in a disease accompanied by impaired glucose tolerance containing an antibody that specifically recognizes ITIH1.

In one embodiment, the present invention is directed to a pharmaceutical composition containing the antibody or antigen-binding fragment according to the present invention as an active ingredient, and the pharmaceutical composition is formulated along with a pharmaceutically acceptable carrier, and optionally, an excipient or stabilizer.

In another embodiment, the pharmaceutical composition according to the present invention is formulated along with a pharmaceutically acceptable carrier, and optionally, an excipient or stabilizer.

The disease accompanied by impaired glucose tolerance and the disease accompanied by hyperglycemia include, but are not limited to, metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney diseases, muscle diseases, and dementia. Insulin resistance promotes cell death in the progression of a variety of diseases, which may cause inflammation. Therefore, all of the diseases described above are related to insulin resistance.

In addition, the amelioration in insulin resistance may provide effects of increasing cell viability and improving cell regeneration based on increased sugar utilization, thus providing anti-inflammatory activity.

In addition, the amelioration in insulin resistance enables effective use as a therapeutic agent for diabetes.

As used herein, the term “metabolic disease” or “metabolic syndrome” refers to a group of diseases including risk factors of various cardiovascular diseases and type 2 diabetes. This is a helpful concept that can encompass and explain insulin resistance and various related complicated metabolic abnormalities and clinical features. Metabolic syndrome increases the risk of cardiovascular disease or type 2 diabetes. It has been reported that the number of patients with metabolic syndrome is increasing explosively with the increase in the obese population. Insulin resistance caused by excessive weight and/or obesity is a key determinant of chronic morbidity in cases of energy metabolism abnormalities (diabetes), and induces chronic inflammatory conditions and cardiovascular abnormalities. Therefore, metabolic abnormalities promote the onset of cardiovascular diseases and are a fundamental cause of chronic intractable diseases that increase the risk of fatty accumulation and severe hepatic diseases in liver tissue (Anstee et al., Gastroenterology & Hepatology, 2013, Vol 10:330-344).

Hyperglycemia is classified as prediabetes when the fasting blood glucose level is 5.6 mM to 7 mM (100-126 mg/dl) in the human body, and diabetes when the fasting blood glucose level is 7 mM (126 mg/dl) or higher. When fasting blood glucose is randomly measured, if it exceeds 11.1 mM (200 mg/dl), it is classified as diabetes. However, pathological symptoms due to hyperglycemia are recognizable when the fasting blood glucose level reaches 15 mM to 20 mM (250-300 mg/dl). As used herein, the term “high-concentration glucose” means a concentration sufficient to cause an increase in concentration due to the mechanism identified herein, that is, activation of OGT attributable to reduction of Ga13 and stabilization of ITIH1 protein thereby. For example, the high-concentration glucose is from about 15 mM to about 35 mM, in particular about 25 mM. A normal concentration of glucose corresponds to about 3.9 mM to 7.1 mM (70-130 mg/dl), which is a fasting blood sugar level in humans, but an average fasting blood sugar in normal subjects is about 5.5 mM (100 mg/dl).

As used herein, the term “insulin resistance” refers to the condition in which the action of insulin for lowering postprandial blood sugar is lower than a normal state, resulting in hyperglycemia.

As used herein, increased insulin sensitivity or reduced insulin resistance means the case in which fasting blood glucose is reduced to about 5.5 mM (100 mg/dl) or less from a high blood glucose before administration.

Here, ITIH1, the expression of which is increased in the presence of hyperglycemia, is one of the heavy chains constituting the inter-alpha-trypsin inhibitor complex (IaI), is called “serum-derived hyaluronan-associated protein (SHAP)” and is a protein produced in the liver cells and secreted into the blood. High expression of SHAP is observed at the site of the inflammatory reaction in the body of patients with rheumatoid arthritis or irritable bowel syndrome (inflammatory bowel disease). However, the expression regulation, mechanism, and role of ITIH1 in hyperglycemia or systemic inflammatory and stressful situations other than local inflammatory environments have not been elucidated to date. In the case of a disease or physiological condition, regulation in the blood glucose concentration in the body varies. In particular, in metabolic syndromes accompanied by various stresses or insulin resistance, an excessive increase in blood sugar occurs, and if this situation continues, pathological reactions in the liver and various organs occur, causing tissue damage and dysfunction. Stimulation with a high concentration of glucose is directly related to insulin resistance and glucose toxicity, which can promote O-GlcNAc modification (O-GlcNAcylation) using glucose as a substrate. O-GlcNAcylation is mediated by an OGT enzyme and enables binding to serine/threonine residues of the target protein to induce GlcNAcylation modification, resulting in changes in the amount and function of the target.

As used herein, the term “pharmaceutically acceptable carrier” refers to a physiologically compatible substance, containing, for example, any solvent, dispersion medium, coating agent, antibacterial and antifungal agent, isotonic solution, absorption/resorption delaying agent, or the like. In one embodiment, the carrier used herein is, in particular, a substance suitable for injection and infusion. For example, the pharmaceutically acceptable carrier may contain a sterile aqueous solution or isotonic buffered saline or dispersion and a sterile powder for preparing a sterile injectable solution. Those skilled in the art will be able to select an appropriate formulation depending on the type of active ingredient contained in the composition.

The composition of the present invention may be administered through various routes known in the art, and it will be apparent to those skilled in the art that the administration method and route may vary depending on the desired effect. The antibody or fragment thereof according to the present invention or composition containing the same may be administered by, for example, parenteral administration such as intravenous infusion, bolus injection, or intramuscular or subcutaneous injection. In addition, the composition of the present invention may be formulated in the form of an appropriate pharmaceutically acceptable dosage, e.g., in a hydrated form, e.g., in an aqueous solution, or in a lyophilizate, regardless of the route of administration.

As used herein, the term “treatment” refers to an action that delays the progression of a disease accompanied by impaired glucose tolerance or a disease accompanied by hyperglycemia, or suppresses, alleviates or eliminates physiological changes or symptoms resulting therefrom by administering the antibody or composition of the present invention to a mammal.

As used herein, the term “prevention” refers to an action that avoids or delays the onset of a disease by administering the antibody or composition of the present invention to a mammal compared to when the antibody or composition is not administered.

Accordingly, the antibody or fragment thereof according to the present invention or a composition containing the same may be administered to a subject who has already developed a disease, a subject who is highly likely to develop the disease, and a subject in need of prevention of the disease.

As used herein, the term “subject” refers to humans, non-human primates, and other mammals, in particular, refers to a subject or patient in need of treatment or prevention of a disease accompanied by impaired glucose tolerance or a disease accompanied by hyperglycemia.

The effective dosage and administration period of the antibody or fragment thereof according to the present invention or a pharmaceutical composition containing the same as an active ingredient may vary depending on the desired therapeutic effect in consideration of a specific patient, the type and administration method of the antibody contained in the composition, etc., and should not cause toxicity to the patient. The actual dosage for each patient should be selected in consideration of various factors such as the activity of the composition to be used, the administration route, the administration time, the secretion rate, other drugs used in combination therewith, gender, age, weight, general health, and underlying diseases. In one embodiment, the antibody of the present invention may be administered in an amount of about 1 to 100 mg/kg body weight, for example, about 10, 20, 30, 40, or 50 mg/kg body weight for the treatment or prevention of a disease. In some cases, the antibody may be administered in an amount of up to about 100 mg/kg.

The administration interval of the antibody or fragment thereof according to the present invention or a pharmaceutical composition containing the same as an active ingredient may be administered at an appropriate interval in units of days, weeks, or months in consideration of the half-life of the administered antibody.

In another embodiment, the antibody according to the present invention may be a multimeric antibody, a heterodimeric antibody, a homodimeric antibody, a multivalent antibody, or a single-chain antibody.

In another aspect, the present invention is directed to a method of ameliorating insulin resistance in a disease accompanied by impaired glucose tolerance, the method including administering the antibody or antigen-binding fragment thereof according to the present invention to a subject in need of amelioration of insulin resistance in a disease accompanied by impaired glucose tolerance.

In one embodiment, the disease accompanied by impaired glucose tolerance in the method includes metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney disease, muscle disease, and dementia.

In the method, the type of disease, subject, administration method, etc. are as described above.

Hereinafter, the present invention will be described in more detail with reference to the following examples. These examples are provided merely for illustration of the present invention, and should not be construed as limiting the scope of the present invention.

### Example

Test Method

Animal Test

All animal experiments were performed in accordance with animal experimentation guidelines established by Seoul National University. Animals were kept under an environment of alternating light and dark at a 12-hour cycle, and were bred with free access to feed. In all experiments, male C57BL/6 strain mice were used. In the diet-induced obesity model experiment, 8-12 week old mice were fed a high-lipid diet (60% of the intake of dietary calories derived from lipids) or a normal diet for 5 weeks. In the glucose oral administration model experiment, 10-week-old C57BL/6 mice were fasted overnight, orally administered with glucose (2 g/kg body weight), and then euthanized at the indicated time, and samples were collected therefrom. For the production of Ga13-deficient mice, Gna13^{flox/flox} mice (provided by Professor Stefan Offermanns of Max Planck Institute, Germany) were crossed with transgenic mice expressing the albumin-Cre gene (purchased from Jackson Laboratory) to construct liver-selective Gna13-deficient mice. Gna13^{flox/flox}, which does not express the Cre gene, was used as a control. In the diet-induced obesity model experiment, 8-12 week old mice were fed a high-lipid diet (60% of dietary calories consumed from lipids) or a normal diet for 5 weeks. In the glucose oral administration model experiment, 10-week-old C57BL/6 mice were fasted overnight, orally administered with glucose (2 g/kg body weight), and euthanized at the indicated time, and tissue samples were collected therefrom.

Western Blot

The same amount of protein for each sample was separated depending on the molecular weight by sodium dodecyl sulfate-polyacrylamide gel electrophoresis (SDS-PAGE) and transferred to a nitrocellulose membrane (GE healthcare). The membrane was reacted with a 5% skim milk solution for 1 hour, then reacted with a primary antibody recognizing each protein for at least 12 hours, and then further reacted with HRP-conjugated IgG (Zymed Laboratories) for one hour, and color development was induced using Amersham ECL western blotting detection reagent (GE Healthcare).

Injection of Plasmid into Mice Using Hydrodynamic Injection

A large amount of shRNA (shOGT) or control plasmid (shCon) targeting OGT was obtained, dissolved in PBS in an amount of volume corresponding to 8% of the mouse weight, and then injected into the tail of 8-week-old normal or hepatocyte-selective Gna13-deficient mice. Each mouse was injected with 25 μg of plasmid within 5 seconds.

Insulin Sensitivity Indicator (Akt Phosphorylation) Experiment

Primary hepatocytes were isolated from livers of normal or Ga13-deficient animals that had been fed a normal diet, and then conditioned media, obtained by treatment with ITIH1-targeting siRNA or a control group thereof, were cultured in 313-L1 and C2C12 cell lines, respectively, for 24 hours. Then, the result was treated with insulin (100 nM), a cell homogenate was sampled 15 minutes later, and the degree of Akt phosphorylation as a downstream signal of the insulin receptor was analyzed through an immunochemical method using a phospho-Akt antibody (Cell Signaling Technology, USA). Using the Akt antibody (Cell Signaling Technology, USA) that recognizes the total Akt protein, the expression level of phosphorylated Akt was calibrated based on the expression level of total Akt and quantified.

2-Deoxyglucose Absorption Intake Capacity Test

The content of 2-deoxyglucose absorbed into cells was measured in the cell homogenate through an enzyme reuse amplification method using a kit for measuring glucose absorption capacity according to the manufacturer's instructions.

Glucose Tolerance Test

From the day before the glucose tolerance test, the mice were allowed to freely access drinking water while fasting for about 16 hours. The fasting blood glucose was measured (time 0), the mice were orally administered with glucose at a concentration of 2 g/kg body weight, and the blood glucose concentration was measured using a glucometer (an Accu-Chek active glucose detection apparatus, Roche) from a small amount of blood obtained by forming a small wound on the tail after 15 minutes, 30 minutes, 60 minutes, and 90 minutes had elapsed.

Insulin Tolerance Test

On the day of the insulin tolerance test, the mice were allowed to freely access drinking water while fasting for about 4 to 6 hours. Then, the fasting blood glucose was measured (time 0) and the mice were administered intraperitoneally with insulin at a concentration of 1.5 IU/kg body weight. Blood glucose concentration was measured using glucometer (an Accu-Chek active glucose detection apparatus, Roche) from a small amount of blood obtained by forming a small wound on the tail whenever 30 minutes, 60 minutes, 90 minutes, and 120 minutes had elapsed.

Obtaining Conditioned Medium Samples from Primary Hepatocytes

Primary hepatocytes isolated from mice fed a high-lipid diet for 5 weeks were rinsed with PBS and then cultured using OPTI-MEM™ medium from which serum was removed. Then, the conditioned medium obtained by performing culture for 24 hours was collected, mixed, and centrifuged at 3,000 g for 5 minutes, and the supernatant was collected and stored in a freezer at −80° C. until used in the experiment. For secretome analysis, large amounts of serum proteins (albumin and immunoglobulin) in the conditioned medium were removed using a commercially available immuno-neutralizing adsorbent resin, followed by concentration through centrifugation at 4,800 g at 4° C. for 90 minutes using an AMICON™ ultra tube.

Preparation of Primary Hepatocyte-Derived Conditioned Medium Samples for Proteomics Analysis

In order to use the conditioned medium sample obtained from primary hepatocytes for liquid chromatography-mass spectrometry, the protein concentration of the conditioned medium was measured using QUICK START™ Bradford 1× dye reagent. Then, the protein fraction (100 mg) was dissolved in 50 mM ammonium bicarbonate and then reduced/alkylated using each of dithiothreitol and iodoacetamide. To lyse the sample by enzymatic reaction, protein and trypsin enzyme were added at a ratio of 50:1 and reacted at 37° C. for 16 hours. The sample subjected to the enzymatic reaction was allowed to flow down in a high-pH liquid phase in a C18 column and divided into 12 fractions.

Production and Characterization of ITIH1 Monoclonal Antibody

Monoclonal antibodies 5D6 and 9E1 against ITIH1 were produced by AbClon Inc. (Seoul, Korea). In brief, human ITIH-1 antigen (SEQ ID NO: 24) produced in HEK293F was purified, 100 to 200 μg of the human ITIH-1 antigen was mixed with an adjuvant (Sigma), the resulting mixture was injected into mice (BALB/c), and blood was collected from mice to determine whether or not antibodies were produced by ELISA. After immunization was performed 2 times, the antibody titer (1:5,000) increased appropriately. Then, the spleen was removed from the immunized mice, and B lymphocytes were isolated therefrom and were then fused with cultured myeloma cells (sp2/0). The fused cells were cultured in a medium supplemented with hypoxanthine, aminopterin, and thymidine (HAT medium), and only cells (hybridoma) fused with myeloma and B lymphocytes were selected and cultured (because B lymphocytes are normal cells and thus die during long-term culture, but myeloma cells are transformed cells and are thus eliminated by HAT selection). Among the obtained hybridoma cells, cells capable of producing an antibody that reacts with an antigen were identified by ELISA. At this time, a process of separating positive cells from negative cells using a limiting dilution method was repeatedly performed on cells that are positive for ELISA (cloning) to produce monoclonal cells (hybridoma) that produce antigen-reactive antibodies. The produced hybridoma cells were stored in a frozen state (1×10⁶ cell/ml).

The epitopes of 5D6 and 9E1 were determined using the PEPPERMAP® epitope mapping kit from AbClon Inc. in accordance with the manufacturer's instructions.

The affinity of 5D6 and 9E1 was determined by AbClon Inc. using an AR2G (Bio-Sensor). The results are as follows.

| | Kon | Kdis | Rmax | KD | Full | Full |
|---|---|---|---|---|---|---|
| Ligand | (1/Ms) | (½) | (nm) | (M) | X² | R² |
| 5D6 | 2.91E+05 | 7.05E−05 | 0.3256 | 2.43E−10 | 0.0186 | 0.9968 |
| 9E1 | 3.34E+05 | 4.45E−05 | 0.9495 | 1.33E−10 | 0.0208 | 0.9996 |

Preparation and Administration of ITIH1 Neutralizing Polyclonal Antibody

The polyclonal antibody targeting ITIH1 was produced by preparing a synthetic peptide (C-DKAREVAFDVE) (SEQ ID NO: 29) predicted from the sequence of mouse ITIH1, polymerizing (conjugating) the synthetic peptide with the KLH moiety to increase antigenicity, and injecting the result into rabbits (immunization), and IgG was purified from the obtained serum. Pre-immune IgG was endogenous IgG obtained from rabbits that did not induce immune response and was administered to the control group. The mice were administered daily with 250 μg of purified ITIH1 polyclonal antibody or pre-immune IgG diluted in PBS for a total of 2 weeks.

Statistical Analysis

Protein bands visualized by immunochemical analysis were quantified using ImageJ software. Data were expressed as mean±standard error, and statistical significance between groups was classified as P<0.05 or P<0.01 using a Student's t-test. A multiple means comparison was performed using ANOVA, followed by the Bonferroni method to calculate statistical significance.

### Example 1. Establishment of System Mimicking Hyperglycemia Stimulation Using Hepatocytes Derived from Liver-Selective Ga13 (Upstream Regulator of ITIH1)-Deficient Mice

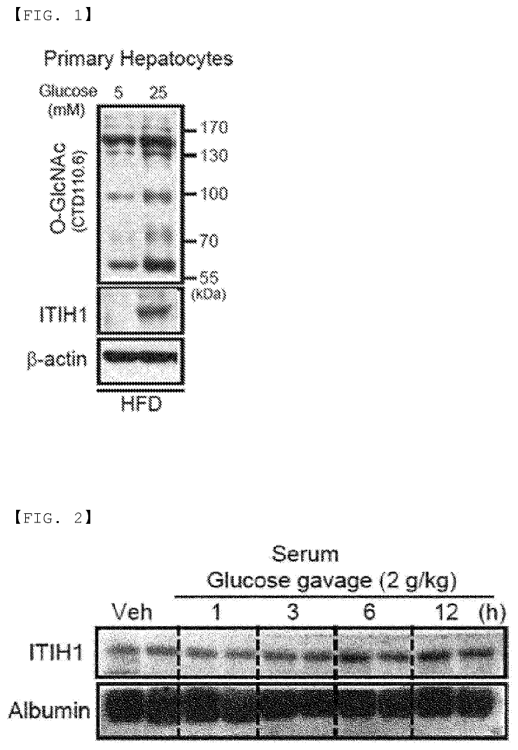

In the present invention, primarily cultured hepatocytes were isolated from liver-selective Ga13 (upstream regulator of ITIH1)-deficient mice and were then cultured for 12 hours in a culture medium containing a low concentration (euglycemia) or a high concentration (hyperglycemia) of glucose to devise a cell-based assay mimicking hyperglycemic stimulation (FIG. 1). At this time, the O-GlcNAc modification (CTD110.6 clone) showing increased activity in a hyperglycemic environment was increased by stimulation with high-concentration glucose compared to stimulation with low-concentration glucose, and the expression of ITIH1, a key target, was remarkably increased. These results indicate that a cell-based assay that mimics a high blood glucose situation in a living organism was successfully established, which indicates that this test method can be used to determine the efficacy of small molecule compound candidates targeting ITIH1.

### Example 2. Establishment of Animal Model System in which Ga13 (Upstream Regulator of ITIH1) is Selectively Deficient in the Liver

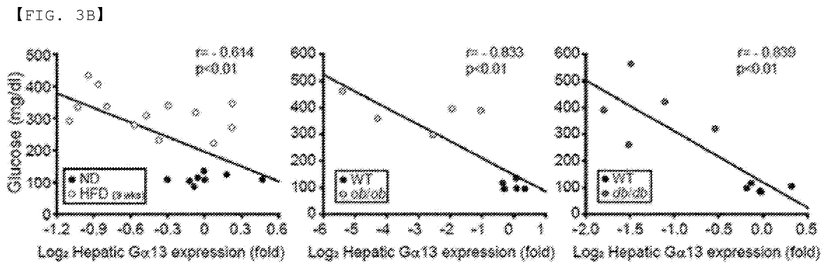

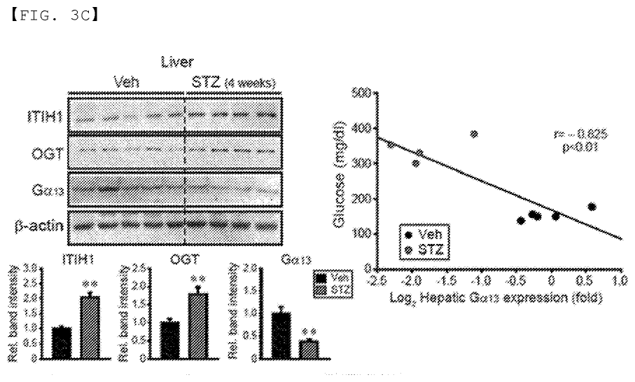

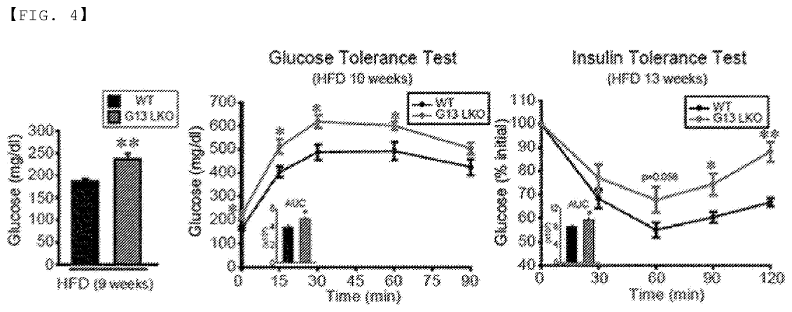

Based on the result showing that Ga13 expression is reduced in a hepatocyte-selective manner in various hyperglycemic conditions (FIG. 3), hepatocellular-selective Ga13-deficient animals having a condition similar to hyperglycemia (or impaired glucose tolerance) were constructed.

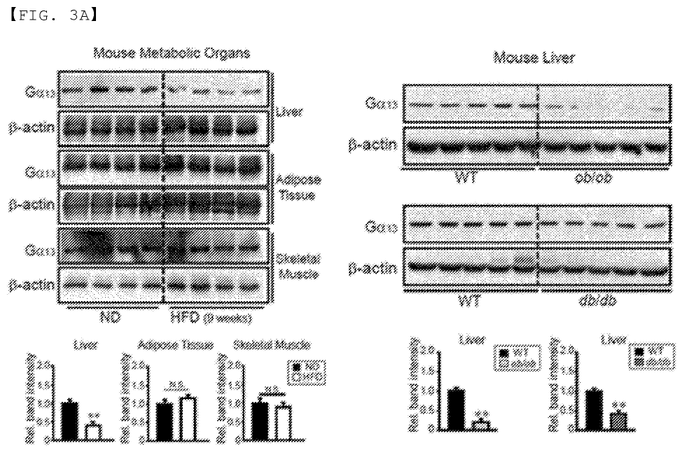

An experimental animal model for verifying the efficacy of a substance used in the cell-based assay proposed herein was suggested. In this example, after a single application of acute hyperglycemic stimulation, ITIH1 production and changes in secretion thereof were observed. The results are shown in FIG. 2. 6 hours after oral administration (2 g/kg) of glucose to normal mice or Ga13-deficient mice (Ga13LKO), the content of ITIH1 in the liver tissue and serum was measured. Similar to the results of the primary hepatocyte culture experiment shown in FIG. 1, upon application of glucose stimulation, the content of ITIH1 in the liver tissue and blood of mice increased, and the expression of ITIH1 increased in Ga13 gene-deficient mice compared to normal mice, even in the absence of glucose stimulation. When glucose stimulation was applied thereto, the ITIH1 content was very high. This is an animal test method using an ITIH1 target proposed in the present invention, which is effective and remarkably improved compared to a conventional model to which a high-lipid diet is administered for 12 to 16 weeks.

### Example 3. Correlation of Changes in Liver Ga13 Expression with Blood Glucose in Various Hyperglycemic Animal Models

In this invention, it was found that the expression of Ga13, which was identified as an upstream regulator of ITIH1, was reduced in the liver tissue of a hyperglycemic experimental animal model induced with a high-lipid diet (left) and a genetically modified mouse model induced with hyperglycemia and obesity (right) (FIG. 3A). The decrease in Ga13 expression in liver tissue was analyzed using immunoblot analysis when C57BL/6 mice were fed a high-fat diet configured such that 60% of all calories consumed were derived from lipids for 9 weeks. There was no change in Ga13 expression in adipose tissue or skeletal muscle isolated from the same mouse (left). In addition, in liver tissue of a transgenic mouse model lacking an appetite-suppressing center, the expression of Ga13 was remarkably decreased in hyperglycemia-induced mice (obob, dbdb) compared to normal mice. Pearson correlation analysis was performed to analyze the relationship between decreased liver Ga13 expression and hyperglycemia in various diabetes models using experimental animals. As a result, hepatic Ga13 expression and blood glucose showed a remarkably high correlation in all hyperglycemia models (FIG. 3B). These results indicate that the expression of Ga13 in the liver is reduced in multiple conditions accompanied by hyperglycemia, and is directly correlated with hyperglycemia.

### Example 4. Increased ITIH1 Concomitant with Hepatic Ga13 Expression Decrease in Hyperglycemic Animal Model

In this example, a hyperglycemia experimental animal model induced as a type 1 diabetes-like model was constructed, and the expression changes of Ga13 and ITIH1 suggested in the present invention were observed. In the present invention, streptozotocin was administered intraperitoneally to 10-week-old C57BL/6 mice at a dose of 50 mg/kg (citrate buffer, pH 4.5) once a day for a total of 5 consecutive days. After 4 weeks, the mice were euthanized to obtain a sample. As a result, the expression of Ga13, an upstream regulator of ITIH1, was decreased, similar to other hyperglycemia models, and the expression of ITIH1 was remarkably increased (FIG. 3C, left). Statistical significance was also verified through Pearson correlation analysis between liver Ga13 expression and blood sugar (FIG. 3C, right). This suggests that the expression of ITIH1 increased along with the decrease in liver Ga13 expression in various hyperglycemic animal models accompanied by diabetes and that this increased ITIH1 expression has a strong negative correlation with actual blood glucose.

### Example 5. Identification of Mechanism by which Increase in OGT Resulting from Decrease in Ga13 in Hyperglycemic State Increases Stability of ITIH1

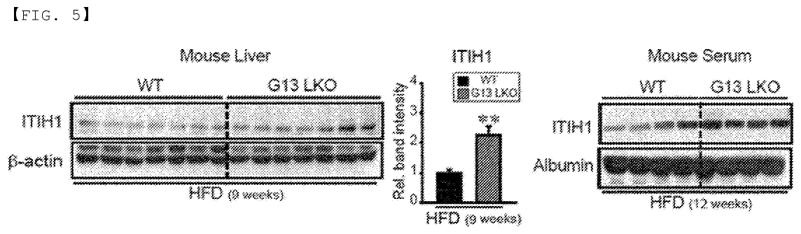

Ga13-selective hepatocyte-deficient mice were constructed, glucose tolerance and insulin tolerance tests were performed on mice that had been fed a high-lipid diet for 9 to 13 weeks, and the results are shown in FIG. 4. In the mice that had been fed a high-lipid diet, fasting blood glucose was significantly increased in hepatocyte-selective Ga13-deficient mice compared to normal mice (left). Additionally, when the glucose tolerance test (middle) and the insulin tolerance test (right) were performed, it was found that Ga13-deficient mice had lower glucose metabolism capability compared to normal mice.

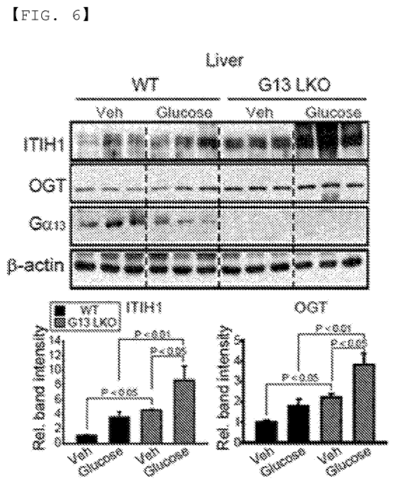

The expression of ITIH1 in liver tissue and sera of normal or hepatocyte-selective Ga13-deficient mice fed a high-lipid diet was analyzed using an immunochemical method. The results are shown in FIG. 5. It can be seen therefrom that the expression of ITIH1 was remarkably increased in the livers and sera of Ga13-deficient mice compared to normal mice.

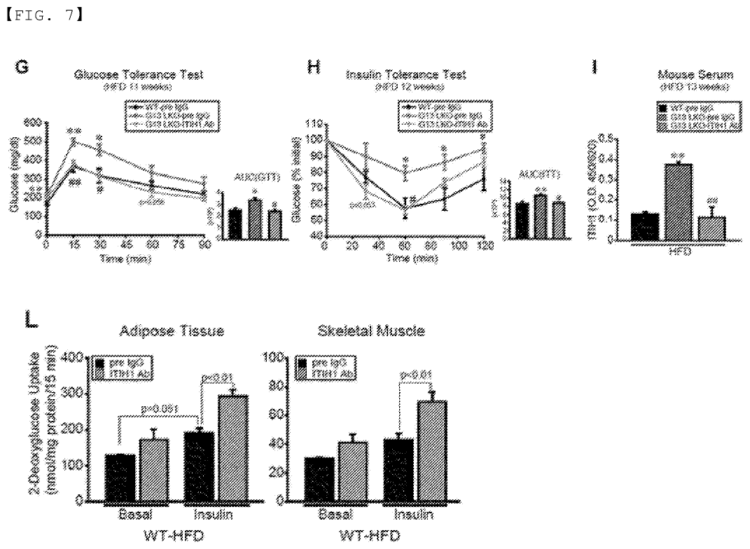

Liver samples were obtained 6 hours after oral administration of high-concentration glucose (2 g/kg body weight) to normal or hepatocyte-selective Ga13-deficient mice, and expression of the corresponding targets was analyzed using an immunochemical method. The results are shown in FIG. 6. When a high concentration of glucose was administered to normal mice, Ga13 decreased, but the expression of ITIH1 and OGT increased, and expression thereof significantly increased in the livers of Ga13-deficient mice.

### Example 6. Effect of Antibody Administration in Hyperglycemic Animal Model

A polyclonal antibody recognizing ITIH1 at a concentration of 250 μg/kg body weight daily for the last 2 weeks was intraperitoneally administered to the hyperglycemic animal model prepared in Example 2, that is, to normal or hepatocyte-selective G13-deficient mice, while providing a high-lipid diet to the mice for 11 to 13 weeks, and then glucose and insulin tolerance tests were performed (FIG. 4). The result showed that the glucose and insulin tolerance of G13-deficient mice was reduced compared to normal mice. As a result of administration of a polyclonal antibody neutralizing ITIH1, it was verified that glucose and insulin tolerance was significantly ameliorated (top). In addition, when the glucose absorption capability was analyzed in white fat and skeletal muscle tissues of normal mice fed a high-lipid diet, it was found that the glucose absorption capability of the peripheral tissues of the mice administered with the ITIH1 polyclonal antibody was significantly improved (bottom).

1. An antibody or antigen-binding fragment thereof specifically recognizing an inter-alpha trypsin inhibitor heavy chain 1 (ITIH1),
wherein the antibody comprises a light-chain variable region comprising complementarity-determining regions CDRL1, CDRL2, and CDRL3 set forth in SEQ ID NOS: 1, 2, and 3, respectively, and a heavy-chain variable region comprising complementarity-determining regions CDRH1, CDRH2, and CDRH3 set forth in SEQ ID NOS: 4, 5, and 6, respectively, or
a light-chain variable region comprising complementarity-determining regions CDRL1, CDRL2, and CDRL3 set forth in SEQ ID NOS: 7, 8, and 9, respectively, and a heavy-chain variable region comprising complementarity-determining regions CDRH1, CDRH2, and CDRH3 set forth in SEQ ID NOS: 10, 11, and 12, respectively.

2. The antibody or antigen-binding fragment thereof according to claim 1, wherein the antibody comprises
a light-chain variable region set forth in SEQ ID NO: 13 and a heavy-chain variable region set forth in SEQ ID NO: 14, or
a light-chain variable region set forth in SEQ ID NO: 15 and a heavy-chain variable region set forth in SEQ ID NO: 16.

3. The antibody or antigen-binding fragment thereof according to claim 1, wherein the antibody is a monoclonal antibody, a chimeric antibody, a humanized antibody, or a human antibody.

4. The antibody or antigen-binding fragment thereof according to claim 1, wherein the antibody is a multimeric antibody, a heterodimeric antibody, a homodimeric antibody, a multivalent antibody, or a single-chain antibody.

5. The antibody or antigen-binding fragment thereof according to claim 1, wherein
an epitope of ITIH1 recognized by the antibody comprises at least one of polypeptides set forth in SEQ ID NO: 17, SEQ ID NO: 18, SEQ ID NO: 19, SEQ ID NO: 20, or SEQ ID NO: 21, for the antibody comprising the light-chain variable region comprising complementarity-determining regions CDRL1, CDRL2, and CDRL3 set forth in SEQ ID NOS: 1, 2, and 3, respectively, and the heavy-chain variable region comprising complementarity-determining regions CDRH1, CDRH2, and CDRH3 set forth in SEQ ID NOS: 4, 5, and 6, respectively, and
the epitope of ITIH1 recognized by the antibody comprises at least one of polypeptides set forth in SEQ ID NO: 22 or SEQ ID NO: 23 for the antibody comprising the light-chain variable region including complementarity-determining regions CDRL1, CDRL2, and CDRL3 set forth in SEQ ID NOS: 7, 8, and 9, respectively, and the heavy-chain variable region including the complementarity-determining regions CDRH1, CDRH2, and CDRH3 set forth in SEQ ID NOS: 10, 11, and 12, respectively.

6. The antibody or antigen-binding fragment thereof according to claim 2, wherein
an epitope of ITIH1 recognized by the antibody comprises at least one of polypeptides set forth in SEQ ID NO: 17, SEQ ID NO: 18, SEQ ID NO: 19, SEQ ID NO: 20, or SEQ ID NO: 21 for the antibody comprising the light-chain variable region set forth in SEQ ID NO: 13 and the heavy-chain variable region set forth in SEQ ID NO: 14, and
the epitope of ITIH1 recognized by the antibody comprises at least one of polypeptides set forth in SEQ ID NO: 22 or SEQ ID NO: 23 for the antibody comprising the light-chain variable region set forth in SEQ ID NO: 15 and the heavy-chain variable region set forth in SEQ ID NO: 16.

7. The antibody or antigen-binding fragment thereof according to claim 1, wherein the antibody or antigen-binding fragment thereof specifically recognizes human- or mouse-derived ITIH1.

8. A nucleic acid encoding the antibody or antigen-binding fragment thereof according to claim 1.

9. The nucleic acid according to claim 8, wherein the nucleic acid encoding the light-chain variable region of the antibody has a sequence set forth in SEQ ID NO: 25 or 27, and the nucleic acid encoding the heavy-chain variable region has a sequence set forth in SEQ ID NO: 26 or 28.

10. A vector comprising the nucleic acid according to claim 8.

11. A cell expressing the vector according to claim 7.

12. A pharmaceutical composition for ameliorating insulin resistance in a disease accompanied by impaired glucose tolerance comprising the antibody or antigen-binding fragment according to claim 1.

13. The pharmaceutical composition according to claim 12, wherein the disease accompanied by impaired glucose tolerance comprises metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney disease, muscle disease, or dementia.

14. The pharmaceutical composition according to claim 12, wherein the pharmaceutical composition has effects of increasing cell viability and improving cell regeneration and thus providing anti-inflammatory activity based on an increase in sugar utilization.

15. The pharmaceutical composition according to claim 12, wherein the pharmaceutical composition is used as a therapeutic agent for diabetes.

16. A method of ameliorating insulin resistance in a disease accompanied by impaired glucose tolerance, comprising administering the antibody or antigen-binding fragment thereof according to claim 1 to a subject in need of amelioration of insulin resistance in the disease accompanied by impaired glucose tolerance.

17. The method according to claim 16, wherein the disease accompanied by impaired glucose tolerance comprises metabolic syndromes, type 1 diabetes mellitus, type 2 diabetes mellitus, diabetic nephropathy, inflammatory bowel diseases including Crohn's disease or ulcerative colitis, obesity, hyperlipidemia, fat hepatitis, steatohepatitis, liver fibrosis or cirrhosis, kidney disease, muscle disease, or dementia.
